(19) United States
(12) Patent Application Publication
(10) Pub. No.: US 2021/0166378 A1
(43) Pub. Date: Jun. 3, 2021

(54) MEDICAL IMAGE ANALYSIS SYSTEM, STORAGE MEDIUM, AND MEDICAL IMAGE ANALYSIS METHOD

(71) Applicant: Konica Minolta, Inc., Tokyo (JP)

(72) Inventors: Nodoka IIDA, Tokyo (JP); Satoshi KASAI, Tokyo (JP)

(21) Appl. No.: 17/108,056

(22) Filed: Dec. 1, 2020

(30) Foreign Application Priority Data
Dec. 2, 2019 (JP) 2019-217677

(51) Int. Cl. G06T 7/00; G16H 50/30; A61B 5/08; A61B 5/00

(52) U.S. Cl. CPC G06T 7/0012; G16H 50/30; A61B 5/08; G06T 2207/20084; G06T 2207/30061; G06T 2207/20081; A61B 5/743

(57) ABSTRACT

Provided is a medical image analysis system including: an acquirer that acquires a medical image obtained by imaging of a patient to be diagnosed; an estimator that estimates an index value indicating a respiratory function from the acquired medical image; and a hardware processor that causes an output unit to output the estimated index value.

### CROSS-REFERENCE TO RELATED APPLICATIONS

The entire disclosure of Japanese Patent Application No. 2019-217677 filed on Dec. 2, 2019 is incorporated herein by reference in its entirety.

### BACKGROUND

### Technological Field

The present invention relates to a medical image analysis system, a storage medium, and a medical image analysis method.

### Description of the Related Art

A spirometry test is commonly used to diagnose respiratory diseases such as chronic obstructive pulmonary disease (COPD) and detect disease conditions. In the test, functions of inhalation, exhalation, and oxygen uptake are measured by actual breathing of a patient.

For example, a “lung age” calculated from measurements of spirometry is used as lung function information used for early detection and prevention of COPD (WO 2014/097449 A1). The lung age, an index of respiratory functions, is calculated from height, FEV1, and sex. The system of lung age calculation may be included in a spirometer used for tests.

### SUMMARY

However, a respiratory function test using a spirometer is not easy and simple, requiring specialized equipment. The test is thus not usually performed on patients without symptoms of COPD. The respiratory function tests are usually performed when a patient is present with a subjective symptom or when the necessity is recognized in an imaging test. Thus, there are not many cases of measuring the lung age of a healthy person, and symptoms may get worse before a respiratory function test using a spirometer is performed or the lung age is measured, making it difficult to develop early detection and prevention of COPD.

Further, repeated actions of deep inhalation and exhalation on a spirometer for the respiratory function measurement may be a burden to the aged.

Therefore, it is desired that an index value of the respiratory functions such as the lung age may be acquired without actually using a spirometer.

The present invention has been conceived in view of the problems in the prior art as described hereinbefore, and has objects of developing early detection and prevention of COPD and reducing a burden to patients.

To achieve at least one of the abovementioned objects, according to an aspect of the present invention, a medical image analysis system reflecting one aspect of the present invention includes:

an acquirer that acquires a medical image obtained by imaging of a patient to be diagnosed;

an estimator that estimates an index value indicating a respiratory function from the acquired medical image; and

a hardware processor that causes an output unit to output the estimated index value.

To achieve at least one of the abovementioned objects, according to another aspect of the present invention, a non-transitory storage medium reflecting one aspect of the present invention stores a computer readable program that causes a computer to:

acquire a medical image obtained by imaging of a patient to be diagnosed;

estimate an index value indicating a respiratory function from the acquired medical image; and

cause an output unit to output the estimated index value.

To achieve at least one of the abovementioned objects, according to another aspect of the present invention, a medical image analysis method reflecting one aspect of the present invention includes:

acquiring a medical image obtained by imaging of a patient to be diagnosed;

estimating an index value indicating a respiratory function from the acquired medical image; and

causing an output unit to output the estimated index value.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a system configuration diagram of a medical image analysis system in a first embodiment of the present invention;

FIG. 2 is a block diagram showing a functional configuration of a data management server;

FIG. 3 shows an example of an image management table;

FIG. 4 is a block diagram showing a functional configuration of an image analysis device;

FIG. 5 is a flowchart of a first lung age estimation process;

FIG. 6 shows an example of a lung age display screen;

FIG. 7 shows an example of output of lung age printed by a printer;

FIG. 8 is a flowchart of a second lung age estimation process in a second embodiment;

FIG. 9 shows an example of a lung age display screen;

FIG. 10 shows another example of the lung age display screen;

FIG. 11 shows an example of output of a lung age transition graph printed by a printer;

FIG. 12 shows an example of a lung age display screen in a variation of the second embodiment;

FIG. 13 is a flowchart of a third lung age estimation process in a third embodiment;

FIG. 14 shows an example of a lung age display screen;

FIG. 15 is a flowchart of a fourth lung age estimation process in a fourth embodiment;

FIG. 16 shows an example of a lung age display screen; and

FIG. 17 shows a method of saving past data in which a medical image and a structured report are associated with one another.

### DETAILED DESCRIPTION OF THE EMBODIMENTS

Hereinafter, embodiments of the present invention are described with reference to the drawings. However, the scope of the present invention is not limited to the embodiments and illustrated examples.

### First Embodiment

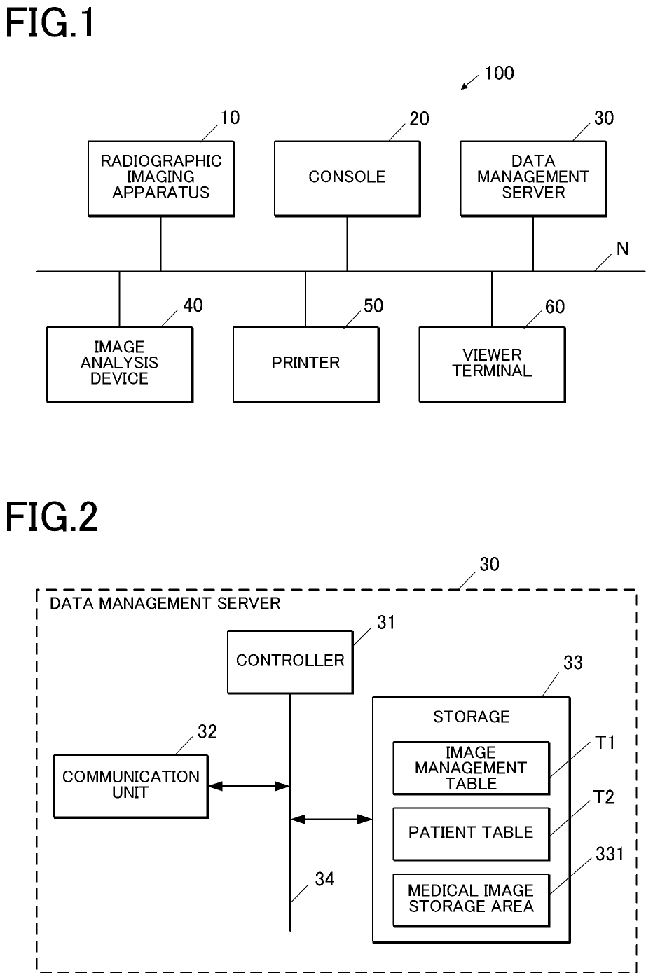

FIG. 1 shows a system configuration of a medical image analysis system 100 in the first embodiment of the present invention. The medical image analysis system 100 includes a radiographic imaging apparatus 10, a console 20, a data management server 30, an image analysis device 40, a printer 50, and a viewer terminal 60, and the components are connected to one another via a communication network N for data communication.

The radiographic imaging apparatus 10, which includes a radiation source, an exposure switch, a radiation detector (flat panel detector (FPD)), and a communication unit, radiographs a subject to generate a medical image(s). The radiographic imaging apparatus 10 radiographs the chest of a patient to be diagnosed to generate a plain radiograph of the chest. The radiographic imaging apparatus 10 may generate a dynamic image by dynamic imaging.

In dynamic imaging, images are obtained as radiation such as X-rays is emitted at predetermined time intervals (pulse emission) or continuously emitted at a low dose without interruption (continuous emission). A cyclic dynamic state of the chest such as change in shape of the lungs by expansion and contraction with breathing and pulsation of the heart is continuously imaged in dynamic imaging, for example. Images obtained by such continuous imaging is called a dynamic image.

The console 20 includes a personal computer (PC) for operation and control of the radiographic imaging apparatus 10 and a specialized equipment.

The data management server 30 is a computer that manages image data of a medical image(s) generated on the radiographic imaging apparatus 10, data concerning lung age, etc. by patient and by test. A picture archiving and communication system (PACS) may be included in the data management server 30, or a cloud server may be used.

In the embodiments hereinafter described, a lung age is used as an example of an index value indicating respiratory function. An index value indicating respiratory function is measurable by a spirometer or calculated from the value measurable by a spirometer.

The lung age, which is calculated from height and forced expiratory volume in one second (FEV1) on the basis of the formulas by sex for the FEV1 defined by Clinical Pulmonary Functions Committee of the Japanese Respiratory Society, is an index of respiratory functions. The lung age is calculated by the following formulas (1) and (2).

Male: Lung age={0.036×height (cm)−1.178−FEV1(L)}/0.028 [FORMULA 1]

Female: Lung age={0.022×height (cm)−0.005−FEV1(L)}/0.022 [FORMULA 2]

In the embodiments, the lung age is calculated by the formulas (1) and (2) above (a measured value of lung age is obtained) in the case where a spirometry test is performed on a patient, mainly for generating training data. In the present invention, the lung age is estimated from a medical image(s) without using the formulas (1) and (2), as described in detail later.

The image analysis device 40 is a computer that estimates the lung age by analysis of image data of a medical image(s) generated on the radiographic imaging apparatus 10.

The printer 50 prints an image(s) on a recording medium such as a paper sheet on the basis of image data for printing.

The viewer terminal 60 is a display unit including a display and an operation interface, and various screens are displayed on the display.

FIG. 2 shows a functional configuration of the data management server 30. The data management server 30 includes a controller 31, a communication unit 32, and a storage 33, and the components are connected by a bus 34.

The controller 31, which includes a central processing unit (CPU) and a random access memory (RAM), centrally controls processing operations of the data management server 30. Specifically, the CPU retrieves various processing programs stored in the storage 33, deploys them in the RAM, and executes various kinds of processing in cooperation with the programs.

The communication unit 32, which includes a network interface, sends and receives data to and from an external device(s) connected via a communication network N. For example, the communication unit 32 receives image data of a medical image(s) obtained by radiographing of a patient by the radiographic imaging apparatus 10. The communication unit 32 sends image data of the medical image and data of the lung age to the image analysis device 40 in response to a request from the image analysis device 40.

The storage 33, which includes a hard disk drive (HDD) and a non-volatile semiconductor memory, stores therein various processing programs, parameters, and files necessary for execution of the programs, etc.

An image management table T1 and a patient table T2 are stored in the storage 33. The storage 33 includes a medical image storage area 331 that stores therein image data of a medical image(s).

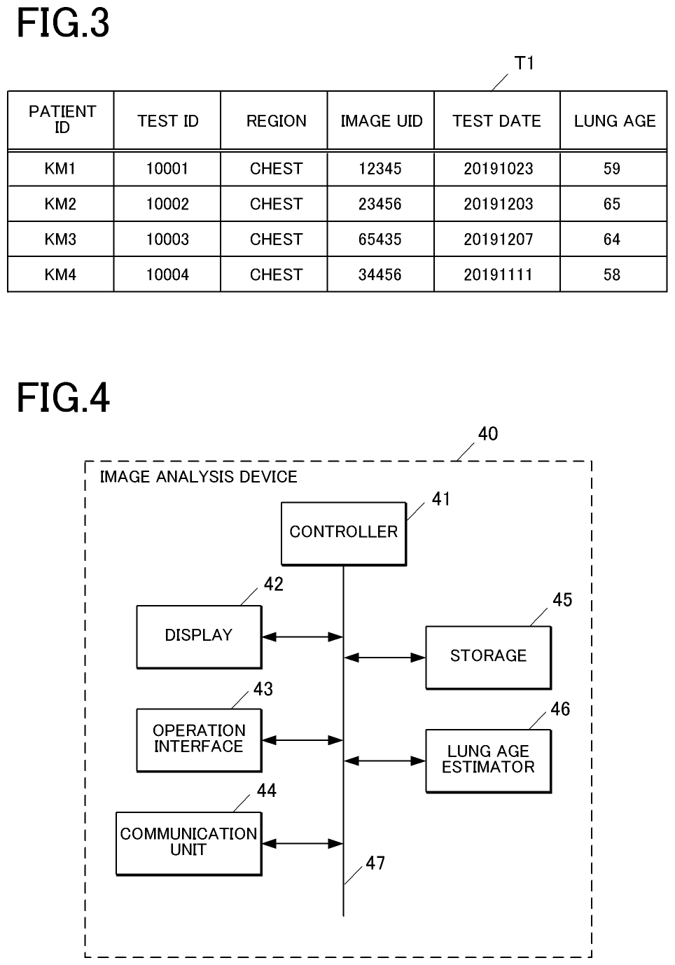

FIG. 3 shows an example of an image management table T1. A patient ID, a test ID, a region, an image UID, a test date, a lung age, etc. are associated with one another by image in the image management table T1.

The patient ID is identification information on a patient.

The test ID is identification information on a test.

The region is a target region of the test by the radiographic imaging apparatus 10.

The image UID is identification information for specifying the medical image.

The test date is a date on which the test (radiographing) is performed by the radiographic imaging apparatus 10.

The lung age is estimated from the medical image specified by the image UID.

A patient ID, name, date of birth, age (actual age), sex, height, weight, etc. are associated with one another in the patient table T2 by patient.

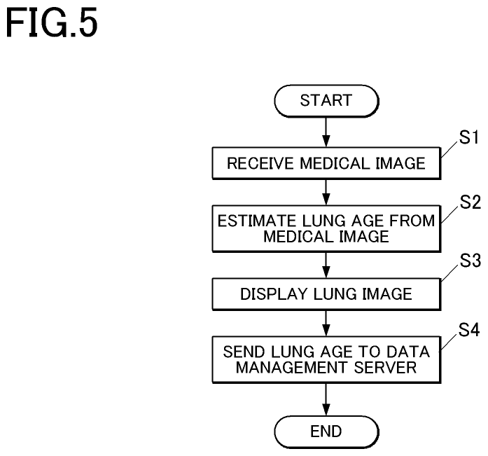

FIG. 4 shows a functional configuration of the image analysis device 40. The image analysis device 40 includes a controller 41 (hardware processor), a display 42, an operation interface 43, a communication unit 44, a storage 45, and a lung age estimator 46, and the components are connected to one another by a bus 47.

The controller 41, which includes a CPU and a RAM, centrally controls processing operations of the image analysis device 40. Specifically, the CPU retrieves various processing programs stored in the storage 45, deploys them in the RAM, and executes various kinds of processing in cooperation with the programs.

Various screens are displayed on the display 42, which includes a monitor such as a liquid crystal display (LCD), according to display signals input from the controller 41.

The operation interface 43, which includes a keyboard with cursor keys, letter and number input keys, various function keys, etc. and a pointing device such as a mouse, outputs operation signals input through keyboard operations or mouse operations to the controller 41. If the operation interface 43 is composed of a touch panel superimposed on the display 42, the operation interface 43 configured as such outputs operation signals according to touch locations of a user's finger, etc. to the controller 41.

The communication unit 44, which includes a network interface, sends and receives data to and from an external device(s) connected via the communication network N. The communication unit 44 receives image data of a medical image(s) obtained by radiographing of a patient to be diagnosed by the radiographic imaging apparatus 10. That is, the communication unit 44 is an acquirer that acquires the medical image(s) obtained by imaging of the patient. The communication unit 44 receives image data of the medical image and data of the lung age from the data management server 30.

The storage 45, which includes an HDD and a non-volatile semiconductor memory, stores therein various processing programs, parameters, and files necessary for execution of the programs, etc.

The lung age estimator 46 estimates the lung age as an index value indicating respiratory function from the acquired medical image(s). The lung age estimator 46 is realized by software processing of the program(s) stored in the storage 45 and the CPU of the controller 41 in cooperation.

The lung age estimator 46 has results of deep learning that uses medical images (radiographs of chest) of patients prepared in advance as input data and the patients' lung ages as output data (single-modal). The lung ages used in deep learning are calculated from the measured values (FEV1) in spirometry tests that patients actually undergo. The lung age estimator 46 estimates the lung age from the medical image of the patient on the basis of the results of the deep learning.

When the medical image of the patient is a still image(s), the deep learning uses still images as the input data, and when the medical image of the diagnosis target patient is a dynamic image(s), the deep learning uses dynamic images as the input data.

The controller 41 causes the lung age estimated by the lung age estimator 46 to be displayed on the display 42 as an output unit.

The controller 41 causes the lung age estimated by the lung age estimator 46 to be printed by the printer 50 as an output unit.

The controller 41 sends the lung age estimated by the lung age estimator 46 to the external device via the communication unit 44 as an output unit (data output).

Next, the operations of the medical image analysis system 100 in the first embodiment are described.

FIG. 5 is a flowchart showing the first lung age estimation process executed by the image analysis device 40.

The controller 41 receives the medical image obtained by radiographing of the chest of the patient from the radiographic imaging apparatus 10 via the communication unit 44 (Step S1). The controller 41 may acquire the medical image of the patient from the data management server 30.

Next, the lung age estimator 46 estimates the lung age from the medical image of the patient (Step S2). Specifically, the lung age estimator 46 outputs the lung age using the medical image received at Step S1 as input data on the basis of the results of the deep learning.

Next, the controller 41 may cause the estimated lung age to be displayed on the display 42 (Step S3).

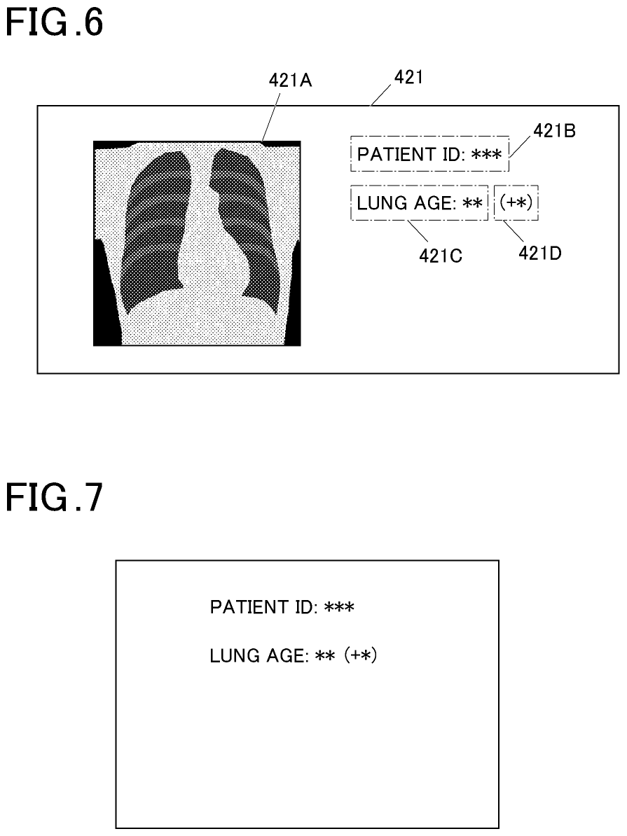

FIG. 6 shows an example of the lung age display screen 421 displayed on the display 42. The lung age display screen 421 includes a medical image display area 421A, a patient ID display area 421B, a lung age display area 421C, and a display area of gap from actual age 421D.

The medical image of the patient is shown in the medical image display area 421A.

The patient ID of the patient is shown in the patient ID display area 421B.

The lung age estimated from the medical image of the patient is shown in the lung age display area 421C.

The value obtained by subtracting the actual age from the lung age of the diagnosis target patient is shown in the display area of gap from actual age 421D.

The controller 41 may cause the printer 50 to print the lung age estimated by the lung age estimator 46. FIG. 7 shows an example of output of the lung age printed by the printer 50.

Next, the controller 41 sends the lung age estimated from the medical image to the data management server 30 via the communication unit 44 (Step S4).

In the data management server 30, the controller 31 stores the image data of the medical image of the patient received from the radiographic imaging apparatus 10 in the medical image storage area 331 of the storage 33, and stores the data concerning the medical image (patient ID, test ID, region, image UID, test date, lung age, etc.) in the image management table T1 in the storage 33. The lung age stored in the image management table T1 is received from the image analysis device 40. The image data of the medical image of the patient may be received from the image analysis device 40. The data of the medical image, the lung age, etc. is accumulated in the data management server 30 as described above.

The first lung age estimation process ends here.

As described hereinbefore, according to the first embodiment, as the lung age (index of respiratory function) is estimated from the medical image obtained by imaging of the patient, the early detection and prevention of the COPD may be developed, and the burden to the patient may be reduced. Conventionally, the lung age is estimated (calculated) by a pulmonary test (spirometry test) that a patient with COPD or a patient with suspected COPD undergoes. However, according to the first embodiment, the lung age may be estimated from a radiograph of the chest imaged by the radiographic imaging apparatus 10 in a periodical health check-up, etc., making it possible to expect early detection of COPD in an environment where spirometers are not usually used.

### Second Embodiment

Next, the second embodiment of the present invention is described.

The medical image analysis system in the second embodiment is configured similarly to the medical image analysis system 100 in the first embodiment. Thus, FIGS. 1 to 4 are referred to, but the configurations thereof are not described. Hereinafter, configurations and processes specific to the second embodiment are described.

In the second embodiment, the lung age estimator 46 of the image analysis device 40 has results of deep learning that uses medical images and clinical data of patients prepared in advance as input data and lung ages of the concerning patients as output data (multi-modal). The lung age estimator 46 estimates the lung age from the medical image and clinical data of the patient on the basis of the results of the deep learning.

The clinical data includes at least one of sex, actual age, height, weight, blood pressure, pulse, and SpO₂.

For example, multimodal data is learned by deep learning, in which image data of the medical images and numerical data of the actual ages and blood pressures of patients prepared in advance as input data are associated with the lung ages measured by spirometry tests on the concerning patients as output data (labeled). This makes it possible to estimate the lung age by integrating the feature values of image data and numerical data, improving the accuracy of estimation.

The controller 41 causes the lung age estimated by the lung age estimator 46 to be displayed on the display 42 as an input unit.

The controller 41 causes all or part of the lung age(s) estimated from the past medical image(s) of the patient and the lung age estimated from the latest medical image of the patient to be displayed on the display 42.

The controller 41 causes a graph showing all or part of the lung ages estimated from the past medical images of the patient and the lung age estimated from the latest medical image of the patient to be displayed on the display 42.

The controller 41 causes all or part of the past medical images of the patient and the latest medical image of the patient to be displayed on the display 42.

The controller 41 detects an abnormality by the lung age estimated by the lung age estimator 46. That is, the controller 41 functions as an abnormality detector.

Next, the operations of the medical image analysis system in the second embodiment are described.

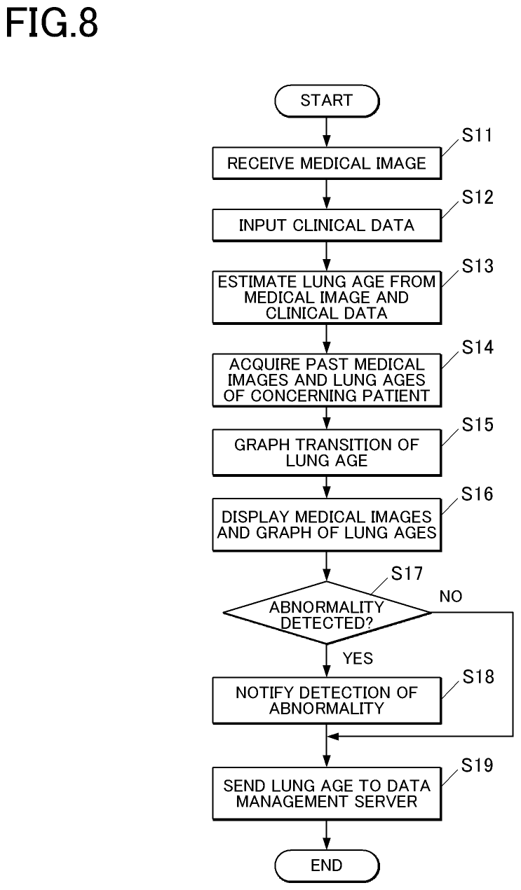

FIG. 8 is a flowchart showing the second lung age estimation process executed by the image analysis device 40.

First, the controller 41 receives a medical image(s) obtained by radiographing of the chest of the patient from the radiographic imaging apparatus 10 via the communication unit 44 (Step S11).

Next, when the user inputs clinical data of the patient from the operation interface 43, the controller 41 acquires the input clinical data (Step S12).

Next, the lung age estimator 46 estimates the lung age from the medical image and clinical data of the patient (Step S13). Specifically, the lung age estimator 46 uses the medical image and clinical data of the patient as input data to output the lung age on the basis of the results of deep learning.

Next, the controller 41 acquires a past medical image(s) and lung age(s) of the patient from the data management server 30 (Step S14). Specifically, the controller 41 sends a request for acquisition of the past medical images and lung ages of the patient to the data management server 30 via the communication unit 44. The request for acquisition includes the patient ID of the patient.

The controller 31 of the data management server 30 refers to the image management table T1 in the storage 33, acquires the image UID(s), the test date(s), the lung age(s), etc. associated with the patient ID of the patient, and specifies the medical image(s) by the image UID(s). The controller 31 sends the past medical images, test dates, and lung ages of the patient to the image analysis device 40 via the communication unit 32.

The controller 41 of the image analysis device 40 acquires the past medical images, test dates, and lung ages from the data management server 30 via the communication unit 44. The controller 41 graphs transition of the lung age (chronological change) over the test dates on the basis of the test dates and lung ages associated with the latest and past medical images (Step S15).

Next, the controller 41 causes the medical images of the patient and the graph of lung ages to be displayed on the display 42 (Step S16).

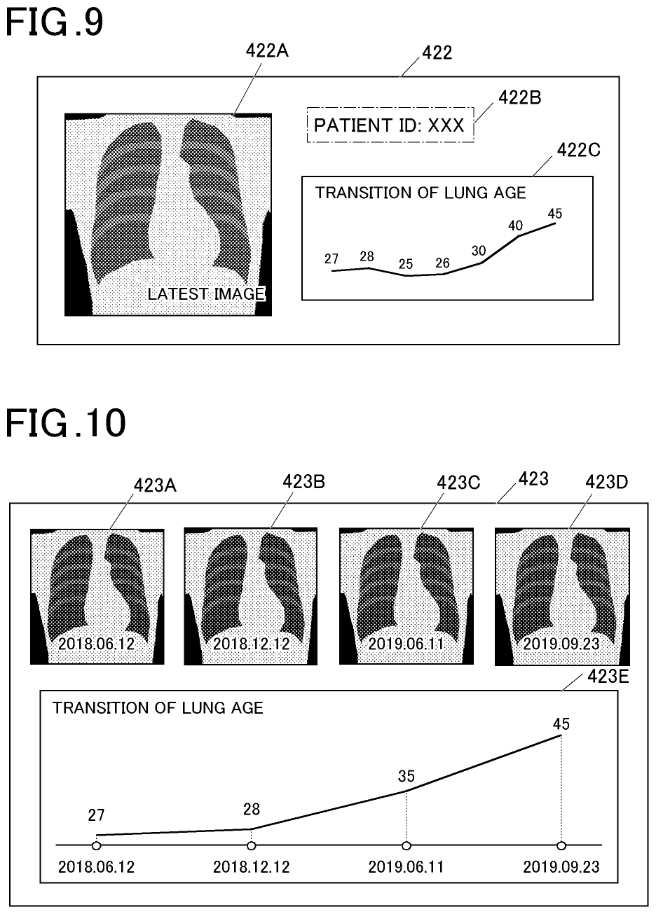

FIG. 9 shows an example of the lung age display screen 422 displayed on the display 42. The lung age display screen 422 includes a medical image display area 422A, a patient ID display area 422B, and a lung age transition graph display area 422C.

The latest medical image of the patient (the medical image received at Step S11) is shown in the medical image display area 422A.

The patient ID of the patient is shown in the patient ID display area 422B.

The graph showing the transition of the lung ages estimated from the latest and past medical images of the patient is shown in the lung age transition graph display area 422C. The horizontal axis represents time, and the vertical axis represents lung age in the graph.

In graphing the lung ages, it is not necessary to use all the lung ages that respectively correspond to the past medical images. The data may be thinned out as long as the transition of lung age can be recognized. A predetermined number of the data values of lung age from the latest may be used.

FIG. 10 shows another example of a lung age display screen 423 displayed on the display 42. The lung age display screen 423 includes a past image display areas 423A to 423C, a latest image display area 423D, and a lung age transition graph display area 423E.

The past medical images of the patient are shown in the past image display areas 423A to 423C.

The latest medical image of the patient is shown in the latest image display area 423D.

The graph showing the transition of the lung ages estimated from the latest and past medical images of the patient is shown in the lung age transition graph display area 423E.

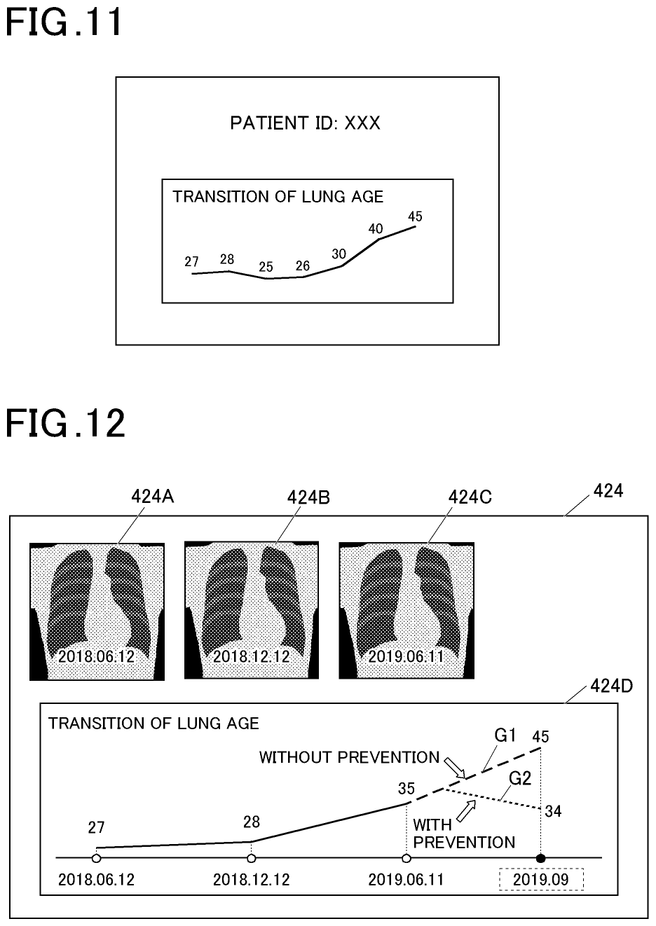

The controller 41 may cause the printer 50 to print the graph of the lung age. FIG. 11 shows an example of the lung age transition graph printed by the printer 50.

Next, the controller 41 determines whether an abnormality is detected on the lung age estimated by the lung age estimator 46 (Step S17). For example, the controller 41 determines that an abnormality is detected if the lung age estimated at Step S13 is older than the actual age by a predetermined value, or if the lung state is rapidly getting worse judging from the transition of the lung ages including the past (the extent of deterioration is greater than a predetermined value).

If an abnormality is detected (Step S17; YES), the controller 41 notifies detection of an abnormality (Step S18). Specifically, the controller 41 causes a notification of detection of an abnormality to be displayed on the display 42. As the notification of detection of an abnormality, the controller 41 may show the estimated lung age itself or the difference value from the actual age so as to indicate the abnormality, or the displayed value (determined as an abnormality) may be highlighted in a different color or bold. The controller 41 may show the extent of abnormality by other indexes such as urgency or by level. A case with a larger extent of abnormality may be listed preferentially at the top of the patient list so as to be diagnosed first.

If an abnormality is not detected after Step S18 or at Step S17 (Step S17; NO), the controller 41 sends the lung age estimated from the medical image to the data management server 30 via the communication unit 44 (Step S19).

In the data management server 30, the controller 31 stores the image data of the latest medical image in the medical image storage area 331 of the storage 33, and stores the data concerning the medical image (patient ID, test ID, region, image UID, test date, lung age, etc.) in the image management table T1 in the storage 33.

The second lung age estimation process ends here.

As described hereinbefore, according to the second embodiment, as the lung age (index of respiratory function) is estimated from the medical image obtained by imaging of the patient, the early detection and prevention of the COPD may be developed, and the burden to the patient may be reduced.

Especially, as the lung ages estimated from the past medical images and the lung age estimated from the latest medical image are shown, the transition of the lung age of the patient may be inspected. The chronological change of the lung age is visualized better by graphing.

### [Variation]

Next, a variation of the second embodiment is described.

In the variation, the controller 41 of the image analysis device 40 causes the predicted future lung age of the patient to be displayed on the display 42. Specifically, the controller 41 uses long short-term memory to predict future transition of one-dimensional chronological data (lung age). The controller 41 uses part of transition of the lung age from data concerning the lung ages (measured values obtained in spirometry tests) collected in advance of patients who did not take preventive measures as input data, predicted values calculated from the part of transition as output data, and deviation of the predicted values from the actual transition (other part of transition not used as input data) for learning to realize prediction of the lung age.

The controller 41 calculates predicted values of lung age in conditions where the patient takes preventive measures to be displayed on the display 42. Specifically, the controller 41 uses part of transition of the lung age from data concerning the lung ages (measured values obtained in spirometry tests) collected in advance of patients who took preventive measures as input data, predicted values calculated from the part of transition as output data, and deviation of the predicted values from the actual transition (other part of transition not used as input data) for learning to realize prediction of the lung age.

FIG. 12 shows an example of a lung age display screen 424 displayed on the display 42 of the image analysis device 40. The lung age display screen 424 includes a past image display areas 424A, 424B, a latest image display area 424C, and a lung age transition graph display area 424D.

The past medical images of the patient are displayed on the past image display areas 424A, 424B.

The latest medical image of the patient is displayed on the latest image display area 424C.

A graph indicating the transition of lung age estimated from the latest and past medical images of the patient with the predicted values is shown in the lung age transition graph display area 424D. A graph G1 indicates predicted values without prevention, and a graph G2 indicates predicted values with prevention.

According to the variation, as not only the transition of lung age (index of respiratory function) of the patient up to the present but also the future predicted values in different conditions (with or without prevention, etc.) are shown, it is possible to draw attention of the patient.

### Third Embodiment

Next, the third embodiment of the present invention is described.

The medical image analysis system in the third embodiment is configured similarly to the medical image analysis system 100 in the first embodiment. Thus, FIGS. 1 to 4 are referred to, but the configurations thereof are not described. Hereinafter, configurations and processes specific to the third embodiment are described.

In the third embodiment, the lung age estimator 46 of the image analysis device 40 estimates the lung age from the medical image and clinical data of the patient based on the results of deep learning that uses the medical images and clinical data of the patients prepared in advance as input data and the lung ages of the concerning patients as output data, as in the second embodiment.

In the third embodiment, the data management server 30 has results of deep learning for estimating the lung age from the medical image and clinical data, similarly to the lung age estimator 46 of the image analysis device 40. The controller 31 learns in advance multiple medical images (chest images) and clinical data as input data and lung ages as output data (labeled). The medical images, clinical data, and lung ages (measured values) (training data) are associated with each other and stored in the storage 33.

The controller 31 of the data management server 30 acquires the medical image or the lung age of the target patient from the image analysis device 40 and searches for cases that have medical images similar to the medical image of the target patient among the medical images (used for deep learning) stored in the storage 33. For example, for extraction of similar images, feature values of deep learning are used. The controller 31 acquires, among the chest images that have been learned in advance, a predetermined number of the images that have feature vectors more similar to those of the chest image (especially the lung region) of the target patient output from the inference of the learned network, as the similar images.

The controller 31 may acquire medical images that are associated with lung ages near the estimated lung age of the target patient as the similar images. The “near” lung age means that a lung age is equal or different within a predetermined value.

The controller 41 of the image analysis device 40 causes the medical images of the cases similar to that of the target patient to be displayed on the display 42. Parameters such as the range of reference for determination of the similarity to the medical image of the target patient and the number of the similar images to be displayed may be preset or designated by the user.

Next, the operations of the medical image analysis system in the third embodiment are described.

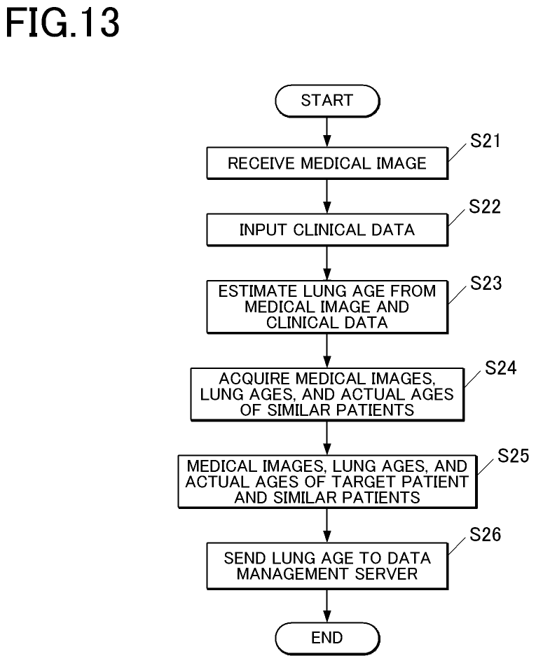

FIG. 13 is a flowchart showing the third lung age estimation process executed by the image analysis device 40.

The process at Steps S21 to S23 is similar to that at Steps S11 to S13 of the second lung age estimation process, and the description thereof is omitted.

Next, the controller 41 acquires the medical image(s) of other patient(s) with lung age(s) and actual age(s) similar to the medical image of the target patient from the data management server 30 (Step S24). Specifically, the controller 41 sends a request for acquisition of medical images with lung ages and actual ages of other patients similar to the medical image of the target patient to the data management server 30 via the communication unit 44. The request for acquisition includes the medical image of the target patient.

The controller 31 of the data management server 30 extracts a predetermined number of the images that have feature vectors more similar to those of the medical image of the target patient among the medical images that have been used for learning. The controller 31 acquires the lung ages associated with the extracted medical images from the training data, and acquires the actual ages corresponding to the extracted medical images from the clinical data (training data). The controller 31 sends the medical images with lung ages and actual ages of other patients similar to the medical image of the target patient to the image analysis device 40 via the communication unit 32.

When the similar images are acquired based on the lung age, a request for acquisition sent from the image analysis device 40 to the data management server 30 includes the lung age estimated from the medical image of the target patient.

The controller 31 of the data management server 30 refers to the image management table T1 in the storage 33, extracts a record(s) including lung age(s) near the lung age of the target patient, acquires a patient ID(s), image UID(s), lung age(s), etc. associated with the record(s) from the patient table T2, and specifies the medical image(s) from the image UID(s). The controller 31 sends the medical images, lung ages, and actual ages of similar patients to the image analysis device 40 via the communication unit 32.

The medical images of similar patients may be extracted from the training data not only when the feature vectors of the medical images are used but also when the lung ages are used.

The controller 41 of the image analysis device 40 causes the medical images, lung ages, and actual ages of the target patient and other patients to be displayed on the display 42 (Step S25).

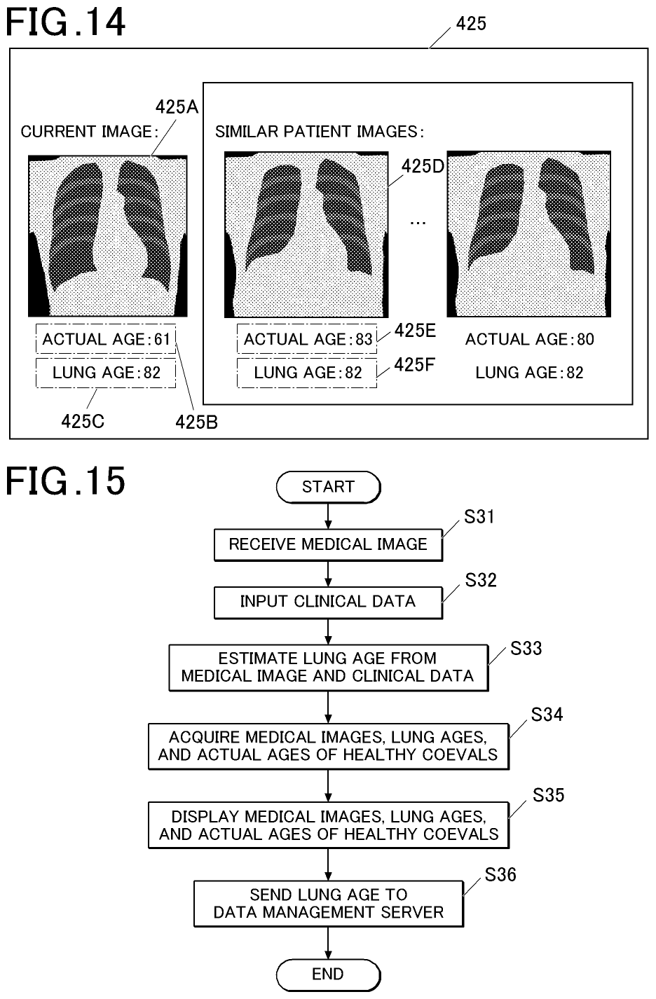

FIG. 14 is an example of a lung age display screen 425 displayed on the display 42. The lung age display screen 425 includes a diagnosis target image display area 425A, an actual age display area 425B, a lung age display area 425C, a similar image display area 425D, an actual age display area 425E, and a lung age display area 425F.

The medical image of the target patient is shown in the diagnosis target image display area 425A.

The actual age of the target patient is shown in the actual age display area 425B.

The lung age of the target patient is shown in the lung age display area 425C.

The medical image(s) of other patient(s) similar to the medical image of the target patient is shown in the similar image display area 425D.

The actual ages of the other patients are shown in the actual age display area 425E.

The lung ages of the other patients are shown in the lung age display area 425E.

Next, the controller 41 sends the lung age estimated from the medical image to the data management server 30 via the communication unit 44 (Step S26). If the lung age of the target patient is sent to the data management server 30 when a medical image(s) of a similar patient(s) is acquired, it is not necessary to send the lung age at this step.

In the data management server 30, the controller 31 stores the image data of the medical image of the target patient in the medical image storage area 331 of the storage 33, and stores the data concerning the medical image (patient ID, test ID, region, image UID, test date, lung age, etc.) in the image management table T1 in the storage 33.

The third lung age estimation process ends here.

As described hereinbefore, according to the third embodiment, as the lung age (index of respiratory function) is estimated from the medical image obtained by imaging of the target patient, the early detection and prevention of the COPD may be developed, and the burden to the patient may be reduced.

As the medical images similar to that of the target patient are shown, it is possible to facilitate diagnosis of the target patient.

As the actual ages of other patients who have a similar chest image (especially lung region) or who are of similar age to the target patient (from top in order of similarity) are compared to the actual age of the target patient, it is possible to help the target patient to take preventive measures especially when the lung age of the patient is older than the actual age. In interpreting the image of the patient, the doctor can refer to the states of the lungs of other patients that have similar respiratory functions in the search results of similar images. This can be assistance to image interpretation.

### Fourth Embodiment

Next, the fourth embodiment of the present invention is described.

The medical image analysis system in the fourth embodiment is configured similarly to the medical image analysis system 100 in the first embodiment. Thus, FIGS. 1 to 4 are referred to, but the configurations thereof are not described. Hereinafter, configurations and processes specific to the fourth embodiment are described.

In the fourth embodiment, the lung age estimator 46 of the image analysis device 40 estimates the lung age from the medical image and clinical data of the patient based on the results of deep learning that uses the medical images and clinical data of patients prepared in advance as input data and the lung ages of the concerning patients as output data, as in the second embodiment.

The controller 41 of the image analysis device 40 causes a medical image(s) of a healthy person(s) who have a predetermined condition(s) in common with the target patient to be displayed on the display 42. The predetermined condition includes age (generation), sex, and height. For example, the “similar age” means that the actual ages of the target patient and the healthy person are equal or different within a predetermined value (for example, 3 years). The “similar height” means that the heights of the target patient and the healthy person are equal or different within a predetermined value (for example, 3 cm).

The controller 31 of the data management server 30 searches for, for example, medical images of a healthy person(s) of the similar age in response to the request for acquisition from the image analysis device 40. The controller 31 searches for healthy persons of the similar age whose actual age is older than the lung age and the same as or different within a value σ of the actual age of the target patient. The controller 31 specifies medical images associated with the healthy persons of the similar age. Parameters such as the threshold value used for determination of the similar age or the number of the healthy persons of the similar age to be extracted may be preset or designated by the user.

Next, the operations of the medical image analysis system in the fourth embodiment are described.

FIG. 15 is a flowchart showing the fourth lung age estimation process executed by the image analysis device 40.

The process at Steps S31 to S33 is similar to that at Steps S11 to S13 of the second lung age estimation process, and the description thereof is omitted.

Next, the controller 41 acquires medical images, lung ages, and actual ages of the healthy persons of the similar age to the target patient from the data management server 30 (Step S34). Specifically, the controller 41 sends a request for acquisition of the medical images, lung ages, and actual ages of the healthy persons of the similar age to the target patient to the data management server 30 via the communication unit 44. The request for acquisition includes the actual age of the target patient.

The controller 31 of the data management server 30 refers to the “age” field of the patient table T2 in the storage 33, and extracts patients whose age is the same as or different within a value σ of the actual age of the target patient. The controller 31 acquires the lung ages of the extracted patients from the image management table T1 in the storage 33, and patients whose actual age is equal to or older than the lung age is set as healthy persons of the similar age. The controller 31 then refers to the image management table T1 in the storage 33, acquires the image UIDs concerning the healthy persons of the similar age (patient IDs of healthy persons), and specifies the medical images from the image UIDs. The controller 31 sends the medical images, lung ages, and actual ages of the healthy persons of similar age to the target patient to the image analysis device 40 via the communication unit 32.

The controller 41 of the image analysis device 40 causes the medical images, lung ages, and actual ages of the patient and the healthy persons to be displayed on the display 42 (Step S35).

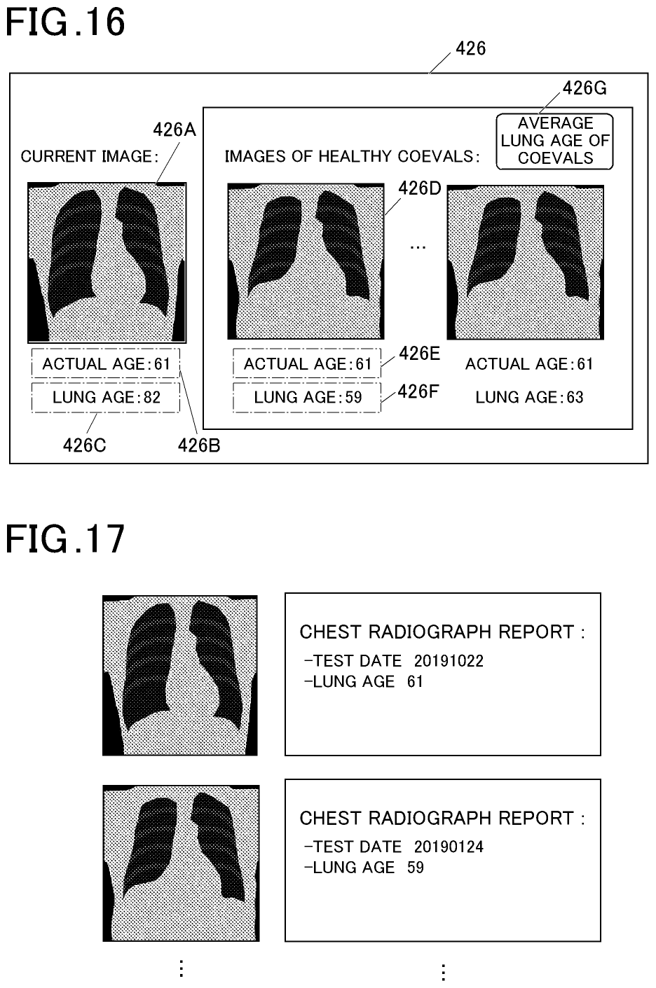

FIG. 16 shows an example of a lung age display screen 426 displayed on the display 42. The lung age display screen 426 includes a diagnosis target image display screen 426A, an actual age display area 426B, a lung age display area 426C, a display area of image of healthy person of similar age 426D, an actual age display area 426E, and a lung age display area 426F.

The medical image of the target patient is shown in the diagnosis target image display area 426A.

The actual age of the target patient is shown in the actual age display area 426B.

The lung age of the target patient is shown in the lung age display area 426C.

The medical images of the healthy persons of the similar age are shown in the display area of image of healthy person of similar age 426D.

The actual ages of the healthy persons of the similar age are shown in the actual age display area 426E.

The lung ages of the healthy person of the similar age are shown in the lung age display area 426F.

The average lung age of the patients of the similar age (426G) may be shown in the lung age display screen 426.

Next, the controller 41 sends the lung age estimated from the medical image to the data management server 30 via the communication unit 44 (Step S36).

In the data management server 30, the controller 31 stores the image data of the medical image of the target patient in the medical image storage area 331 in the storage 33, and stores the data concerning the medical image (patient ID, test ID, image UID, test date, lung age, etc.) in the image management table T1 in the storage 33.

The fourth lung age estimation process ends here.

As described hereinbefore, according to the fourth embodiment, as the lung age (index of respiratory function) is estimated from the medical image obtained by imaging of the target patient, the early detection and prevention of the COPD may be developed, and the burden to the patient may be reduced.

As the medical images, actual ages, and lung ages of the healthy persons of similar age to the target patient are shown, it is possible to help the target patient to take preventive measures especially when the lung age of the target patient is older than the actual age.

As the average value of the lung ages of the patients of similar age is shown, it is possible to compare the value to the lung age of the target patient.

The above description of the embodiments is an example of the medical image analysis system according to the present invention, and is not intended to limit the scope of the invention. The detailed configurations and operations of the components of the system can also be appropriately modified within the scope of the present invention.

For example, processes characteristic to the respective embodiments may be combined with each other.

In the embodiments described above, the lung age is used as an index value indicating the respiratory function to be estimated from the medical image, but the present invention is not limited to this example. An index value other than the lung age may be vital capacity, vital capacity percentage, forced vital capacity, FEV1, or FEV1.0%.

The vital capacity is a volume of air expelled from the lungs after a maximum inhalation.

The vital capacity percentage is a proportion of measured vital capacity to predicted vital capacity (reference) calculated from age and sex.

The forced vital capacity is a volume of air forcibly expelled from the lungs after a maximum inhalation.

The FEV1 is a volume of air expelled from the lungs in the first second of the forced vital capacity.

The FEV1.0% is a proportion (%) of the FEV1 to the forced vital capacity.

In the embodiments described above, the association between the medical images and the lung ages (index values) is managed using a table (the image management table T1), but alternatively, it may be saved as a text in a structured report (SR) of the DICOM images.

FIG. 17 shows a method of saving past data in which a medical image and a structured report are associated with one another.

In the embodiments described above, the lung age (index value) is estimated using results of deep learning obtained by the lung age estimator 46, but the index value indicating respiratory functions may be estimated from the medical images by FEV1 and FEV1% calculated from changes in lung volume obtained from density value changes in front and side images of the lung field in a dynamic image (radiographs sequentially taken).

The estimated data of the lung age may be stored associated with the medical image. Alternatively, the medical image may be stored alone and the lung age may be estimated each time the image is used.

The method of displaying the information concerning the lung age may be selected by the user. For example, the user may select arbitrarily whether the lung ages are shown by values or on a graph, whether the estimated value is included, whether the similar images are shown, whether the medical images of healthy persons of the similar age are shown, etc.

The steps included in each process may be executed on the same device, or on multiple devices.

For example, the console may include the acquirer that acquires the medical image, and the estimator that estimates the lung age (index value indicating respiratory function) from the medical image, and the controller that outputs the estimated lung value via the output unit. In that case, the lung age is estimated from the medical image acquired from the radiographic imaging apparatus 10 on the console 20, the lung age is sent from the console 20 to the viewer terminal 60, and the lung age is displayed on the display of the viewer terminal 60. In this process, the lung age may be sent from the console 20 to the viewer terminal 60 according to user operation via the operation interface of the viewer terminal 60. The data for printing including the lung age may be sent from the console 20 to the printer 50, and the lung age may be printed by the printer 50.

The latest and/or past medical image(s) and lung age(s) may be acquired from the data management server 30 according to the user operation via the operation interface of the terminal device of the patient such as a smartphone, and the acquired medical image and lung age may be displayed on the display of the terminal device. When the estimation result of the lung age, etc. is directly presented to the patient on the smartphone, etc., preventive methods and comments (advice) from the doctor may be shown together.

The data management server 30 may include the acquirer that acquires the medical image, and the estimator that estimates the lung age (index value indicating respiratory function) from the medical image, and the controller that outputs the estimated lung value via the output unit. In that case, the data management server 30 causes the lung age to be displayed on the display of the viewer terminal 60 or the smartphone, or to be printed by the printer 50.

The process of estimation of index values indicating the respiratory functions such as the lung age from the medical image may be executed in a remote location such as a cloud server, and the estimation results may be acquired from the remote location and used.

The training data for deep learning may be medical images taken in medical checkups and lung ages obtained in spirometry tests of the concerning persons.

In the description above, an HDD and a non-volatile semiconductor memory is used as the computer-readable medium storing the programs for executing the operations, but the present invention is not limited to these examples. A portable storage medium such as a CD-ROM can be used as the computer readable recording medium. A carrier wave may be also used as a medium providing the program data via a communication line.

What is claimed is:

1. A medical image analysis system comprising:
an acquirer that acquires a medical image obtained by imaging of a patient to be diagnosed;
an estimator that estimates an index value indicating a respiratory function from the acquired medical image; and
a hardware processor that causes an output unit to output the estimated index value.

2. The medical image analysis system according to claim 1,
wherein the index value is a value measurable by a spirometer or a value calculated from the value measurable by the spirometer.

3. The medical image analysis system according to claim 2,
wherein the index value is a lung age.

4. The medical image analysis system according to claim 1,
wherein the medical image is a plain radiograph of a chest.

5. The medical image analysis system according to claim 1,
wherein the medical image is a dynamic image.

6. The medical image analysis system according to claim 1,
wherein the output unit is a display that displays the estimated index value.

7. The medical image analysis system according to claim 6,
wherein the hardware processor causes the display to display one or more index values respectively estimated from one or more past medical images of the patient in whole or part and an index value estimated from a latest medical image of the patient.

8. The medical image analysis system according to claim 7,
wherein the hardware processor produces a graph of the one or more index values respectively estimated from the one or more past medical images of the patient in whole or part and the index value estimated from the latest medical image of the patient, and causes the display to display the graph.

9. The medical image analysis system according to claim 6,
wherein the hardware processor causes the display to display one or more past medical images of the patient and a latest medical image of the patient in whole or part.

10. The medical image analysis system according to claim 6,
wherein the hardware processor causes the display to display a medical image of a case similar to the medical image of the patient.

11. The medical image analysis system according to claim 6,
wherein the hardware processor causes the display to display a medical image of a healthy person who has a predetermined condition in common with the patient.

12. The medical image analysis system according to claim 6,
wherein the hardware processor causes the display to display a predicted future index value of the patient.

13. The medical image analysis system according to claim 1 comprising:
an abnormality detector that detects an abnormality from the estimated index value.

14. The medical image analysis system according to claim 1,
wherein the estimator estimates the index value from the medical image of the patient based on a learning result of deep learning that uses prepared medical images of patients as input data and prepared index values of the patients as output data.

15. The medical image analysis system according to claim 1,
wherein the estimator estimates the index value from the medical image of the patient to be diagnosed based on a learning result of deep learning that uses prepared medical images and clinical data of patients as input data and prepared index values of the patients as output data.

16. The medical image analysis system according to claim 15,
wherein the clinical data comprises at least one of parameters of sex, actual age, height, weight, blood pressure, pulse, and SpO₂.

17. A non-transitory recording medium that stores a computer readable program that causes a computer to:
acquire a medical image obtained by imaging of a patient to be diagnosed;
estimate an index value indicating a respiratory function from the acquired medical image; and
cause an output unit to output the estimated index value.

18. A medical image analysis method comprising:
acquiring a medical image obtained by imaging of a patient to be diagnosed;
estimating an index value indicating a respiratory function from the acquired medical image; and
causing an output unit to output the estimated index value.
